(19) United States
(12) Patent Application Publication
(10) Pub. No.: US 2006/0271158 A1
(43) Pub. Date: Nov. 30, 2006

(54) Positional locking endoluminal device system

(75) Inventor: Gregory K. Olson, Elk River, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: BOSTON SCIENTIFIC SCIMED, INC., Maple Grove, MN (US)

(21) Appl. No.: 11/138,013

(22) Filed: May 26, 2005

(51) Int. Cl. A61F 2/06

(52) U.S. Cl. 623/1.15

(57) ABSTRACT

A medical device comprises a stent formed out of a multiplicity of interlocking polygonal geometric shape elements connected by connectors.

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks and similar implantable medical devices, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, etc. Stents may be used to reinforce body vessels and to prevent restenosis following angioplasty in the vascular system. They may be self-expanding, such as a nitinol shape memory stent, mechanically expandable, such as a balloon expandable stent, or hybrid expandable.

Stents may be created by methods including cutting or etching a design from a tubular stock, from a flat sheet which is cut or etched and which is subsequently rolled or from one or more interwoven wires or braids.

### BRIEF SUMMARY OF THE INVENTION

Without limiting the scope of the invention a brief summary of some of the claimed embodiments of the invention is set forth below. Additional details of the summarized embodiments of the invention and/or additional embodiments of the invention may be found in the Detailed Description of the Invention below. A brief abstract of the technical disclosure in the specification is provided as well only for the purposes of complying with 37 C.F.R. 1.72. The abstract is not intended to be used for interpreting the scope of the claims. All US patents and applications and all other published documents mentioned anywhere in this application are incorporated herein by reference in their entirety.

This invention contemplates a number of embodiments where any one, any combination of some, or all of the embodiments can be incorporated into a stent and/or a stent delivery system and/or a method of use. At least one embodiment of the invention is directed to a stent constructed out of a multiplicity of interlocking geometric shape elements connected by expandable and contractible elastic members. These geometric shape elements fit together like scales and can be formed into any number of shapes including but are not limited to rectangles, trapezoids, octagons, and hexagons. The stent comprises two or more geometric shape elements connected by a connector.

In at least one embodiment, the connector is an elastic connector. In the context of this application the term “elastic connector” means a connector made of such material that it is both flexible and that it easily rebounds and resumes its original shape or configuration after being stretched, expanded, or deformed.

The geometric shape of the elements and how they are connected determines how these elements can fit together and the number of relative positions these elements can assume. How the elements fit together in turn determines the number of configurations the overall device can assume. In at least one embodiment, the connector must be elastic enough so as to allow for different distances that are required between the elements as they assume different relative positions.

In at least one embodiment, the geometrical shaped elements are polygonal.

At least one embodiment of the invention is directed to a stent made up of two or more geometrically shaped elements that can be bent into a variety of configurations based on the shape of the geometrically shaped elements. A connector pulls the elements together and the elements' particular geometric shape determines how the elements can align relative to each other. As connector tension pulls the elements closely together, specially shaped locking points on the elements fit into each other and cause the elements to become “locked”. The particular shapes of the elements determine how the elements can “lock” together which in turn determines what overall shapes the device can assume. The locking points can be similarly shaped surfaces or edges of the elements laying flush against each other or they can be highly complex locking mechanisms. As the elements lock together they form a stent with strong scaffolding characteristics but because this device is made of multiple parts, the scaffolding strength does not come with the loss of flexibility present in unibody devices.

The geometrically shaped elements can be designed so as to form more than one stent configuration. This can be done by arranging for the elements to fit together into more than one combination. This can be useful for situations where the device needs to change shape or needs to react to different kinds of environmental strain. In cases of different environmental strains, the geometrically shaped elements can be designed so that one or more configurations will be assumed in reaction to low strain, and the one or more other configurations can be assumed in reaction to high strain.

In at least one embodiment, the shaped geometric elements provides superior kinetics for drug delivery because the shape elements provide a greater surface area for drug coatings to interact on than is available on mesh unibody stents.

In at least one embodiment, a stent made out of geometrically shaped elements could be used with balloon expansion stent installation procedure by placing it over a deflated balloon. Upon expansion, the stent will shift from a first state with a short diameter to second expanded state with a larger diameter by shifting from one locked configuration of the geometrically shaped elements to another.

In at least one embodiment, the invention is directed to a stent comprising a plurality of plates. Each plate has an inner surface and an outer surface and a plurality of sides disposed about the periphery of the plate extending between the inner and outer surfaces. At least two plates which are adjacent one another are connected flexibly one to the other to allow for reorientation of the plates relative to one another. Adjacent plates have a first orientation in which one of the sides of one of the plates abuts one of the sides of the plate adjacent thereto and a second orientation in which another of the sides of one of plates abuts the same or different side of the plate adjacent thereto.

In at least one embodiment, each of the plates is adjacent another plate and is flexibly interconnected to at least one plate adjacent thereto so as to allow for reorientation of the plates relative to one another. Each two adjacent plates have a first orientation in which one of the sides of one of the plates abuts one of the sides of the plate adjacent thereto and a second orientation in which another of the sides of one of plates abuts the same or different side of the plate adjacent thereto.

In some embodiments, the invention is directed to the stent in the fully expanded state, the unexpanded state and in any states therebetewen. Typically, the stent will be characterized as having a plurality of discrete expanded states with the stent having a different diameter in each of the discrete expanded states.

The plates may be provided a wide variety of shapes including polygonal and desirably, regular polygonal.

Each plate may be flexibly connected to a plate adjacent thereto via a flexible connector made of metal or polymer.

The plates may be arranged substantially helically about a longitudinal axis or in closed cylinders about a longitudinal axis.

In some embodiments, the invention is also directed to a stent comprising a plurality of stent segments, at least two adjacent segments having a plurality of meshing teeth to allow one of the segments to be repositioned relative to the other of the two segments. Desirably, each segment is engaged to at least one adjacent segment via meshing teeth to allow the segment to be repositioned relative to the segment to which it is engaged.

The stent will desirably be expandable between at least two sizes. More desirably, the stent will be discontinuously expandable between more that two different, discrete sizes.

The segments may be arranged helically about a longitudinal axis or may be arranged in serpentine bands which form closed cylinders.

In some embodiments, the invention is also directed to a stent comprising a plurality of stent segments, at least two adjacent segments having a plurality of meshing teeth to allow one of the segments to be repositioned relative to the other of the two segments. In at least one embodiment, each segment is engaged to at least one adjacent segment via meshing teeth to allow the segment to be repositioned relative to the segment to which it is engaged. In at least one embodiment, the stent is discontinuously expandable to a plurality of different, discrete sizes. The segments may be arranged helically about a longitudinal axis or may be arranged in serpentine bands which form closed cylinders.

### BRIEF DESCRIPTION OF THE SEVERAL VIEWS OF THE DRAWINGS

The invention is best understood from the following detailed description when read in connection with accompanying drawings, in which:

FIG. 1 shows a pair of geometrically shaped elements joined by a connector in a first low energy state.

FIG. 2 shows a pair of geometrically shaped elements joined by a connector in a first high energy state.

FIG. 3 shows a pair of geometrically shaped elements joined by a connector in a second high energy state.

FIG. 4 shows a pair of geometrically shaped elements joined by a connector and locked into place in a second low energy state.

FIG. 5 is a side view of a stent created by connecting a multiplicity of geometrically shaped elements where the elements are in the shape of an octagon.

FIG. 6 is a stent created by connecting a multiplicity of geometrically shaped elements where the elements are in the shape of an octagon, as viewed from the proximal side. FIG. 7 is a diagonal view of a stent in an expanded configuration created by connecting a multiplicity of geometrically shaped elements where the elements are in the shape of an octagon.

FIG. 8 is a view of a stent in a high strain configuration created by connecting a multiplicity of geometrically shaped elements where the elements are in the shape of an octagon viewed from the proximal side.

FIG. 9 is a side view of a stent created by connecting a multiplicity of geometrically shaped elements where the elements are in the shape of a rectangle.

FIG. 10 is a stent created by connecting a multiplicity of geometrically shaped elements where the elements are in the shape of a rectangle viewed from the proximal side.

FIG. 11 is a view of a stent in an expanded configuration created by connecting a multiplicity of geometrically shaped elements where the elements are in the shape of a rectangle, as viewed from the proximal side.

FIG. 12 is a schematic of an inventive stent.

FIG. 13 shows portions of the stent of FIG. 12 in greater detail.

FIG. 14a shows an enlargement of FIG. 13.

FIG. 14b shows another enlargement of FIG. 13.

FIG. 15 shows a schematic of the turn with a plurality of strut segments extending therefrom.

### DETAILED DESCRIPTION OF THE INVENTION

The invention will next be illustrated with reference to the figures wherein, unless otherwise indicated, the same numbers indicate similar elements in all figures. Such figures are intended to be illustrative rather than limiting and are included herewith to facilitate the explanation of the apparatus of the present invention.

The invention, in one or more embodiments, is directed to a medical device such as a stent formed from interconnected geometric shaped elements which are capable of being reoriented relative to between a plurality of stable states.

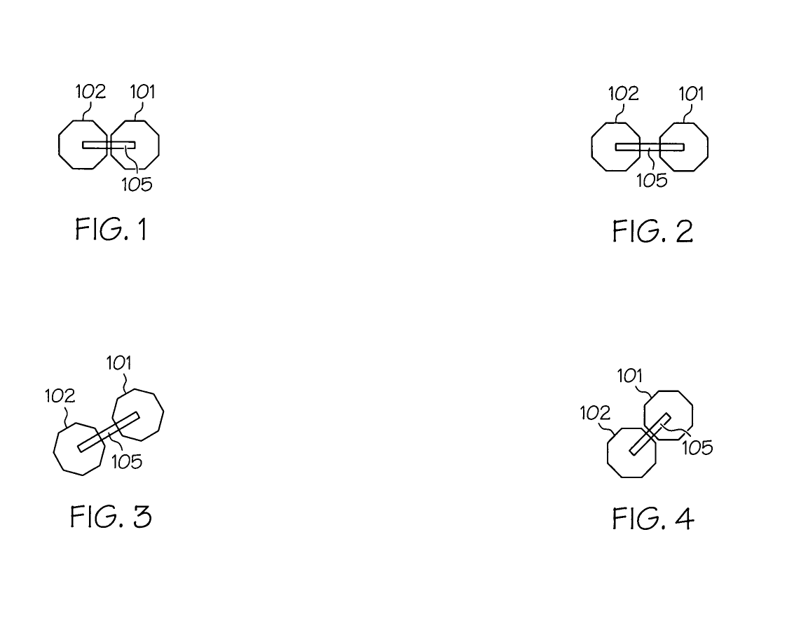

Referring now to FIGS. 1-4, there is shown a first geometrically shaped element 101 and a second geometrically shaped element 102, each in the shape of octagon, which may be present in a stent. First geometrically shaped element 101 and second geometrically shaped element 102 are connected one to the other by an elastic connector 105. FIGS. 1 and 4 show a first and a second, low energy relative position that the elements can assume to fit next to each other with a forty five degree orientation difference between the two and FIGS. 2 and 3 show higher energy transient intermediate configurations through which the geometrically shaped elements must pass in order to transition between the two low energy positions.

More specifically, FIG. 1 illustrates a first locked position. FIG. 2 illustrates the shape elements shifted into in a first high strain position in reaction to an energy input. This energy input could be from any number of sources including a balloon expansion. FIG. 3 shows the shaped elements then pushed into s second high strain position and FIG. 4 shows the elements assuming a second locked position. The second assumed locked position is at a 45 degree angle relative to the first position illustrated in FIG. 1. Once in the second locked position, geometrically shaped elements will remain locked into place by tension from the elastic connector and can only be pushed out of this orientation by an input of energy or force. For this reason the locked positions are referred to as low energy states or strains and the unlocked positions are referred to as high energy states or strains. At least one embodiment of this invention can be constructed out of two or more of these geometrically shaped elements.

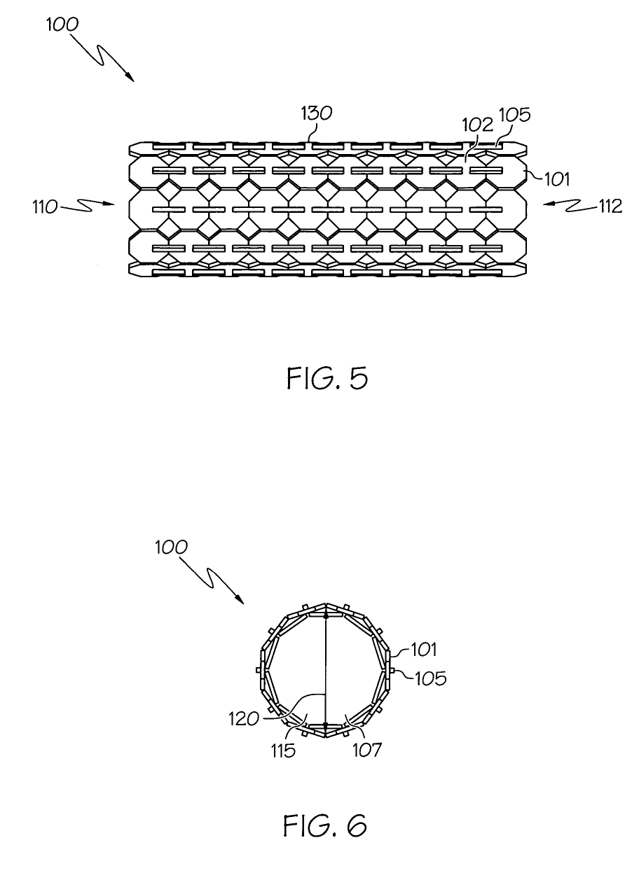

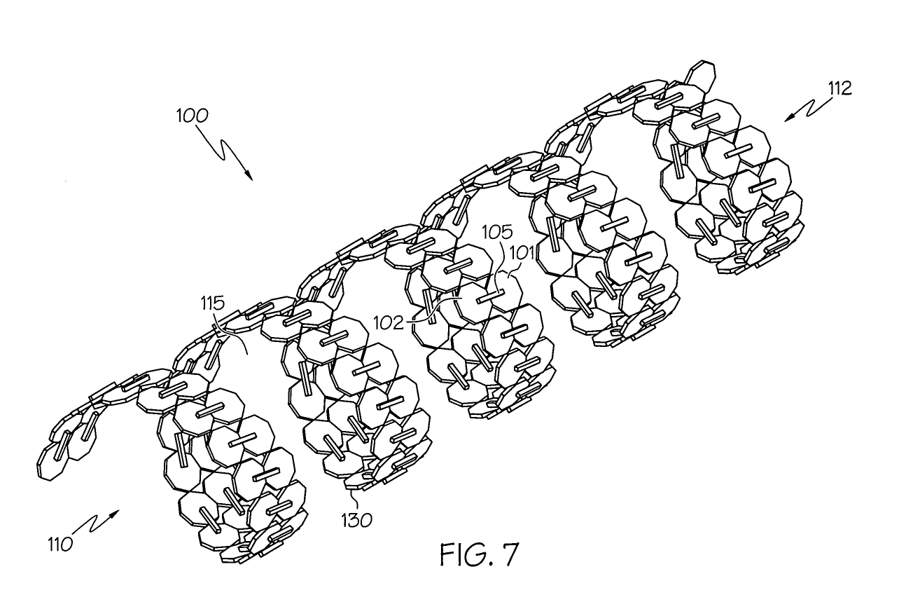

At least one embodiment is constructed out of a plurality of the octagon shaped elements combined to form a stent having a distal end 110, a proximal end 112, a shaft 130, and a hollow interior 115 is illustrated in FIG. 5 and FIG. 6. In these illustrations, the device 100 comprises a number of paired geometric elements each having an outer surface of a first geometrically shaped element 101 an inner surface of a first geometrically shaped element 107 an outer surface of a second geometrically shaped element 102 and an inner surface of a second geometrically shaped element 108 connected by connector 105. In FIGS. 5 and 6 all of the octagon shaped elements are in a zero degree relative position to each other and together form a cylinder shape. The connector 105 exerts a tension between the elements assuring that this configuration has strong scaffolding strength but in the presence of a strong enough force, the connector elasticity provides sufficient flexibility to bend. A lateral view of this configuration can be seen in FIG. 6 where the device diameter 120 and the hollow interior 115 can be seen from the proximal side. In this configuration a view from the distal end would be identical.

While stent 100 of FIG. 1 is shown constructed more octagonal shaped elements, it is within the scope of the invention to use other shaped geometric elements, such as polygons and regular polygons. Examples of other polygonal shapes suitable for use in embodiments of the invention include, but are not limited to, triangles, squares, pentagons, hexagons, septagons, nonagons and decagons, as well as shapes with more sides, all of which shapes may be regular or otherwise. In some embodiments, the geometric elements will have three or more sides. A stent formed of geometric shapes having more sides will be able to be expanded to a greater number of discrete sizes than a stent formed of geometric shapes having fewer sides.

In at least one embodiment, an example of which is illustrated in FIG. 7, some of the octagon shaped elements 101 and 102 are oriented at a forty five degree relative position causing the overall configuration of the device to assume a helical configuration. The relative angle and position of the elements 101 and 102 may be varied to alter the pattern and shape of the helix as desired.

As a helix, the stent 100 may be provided with a greater internal volume 115 than a cylinder made of similar elements. However, because of the tension connector 105 exerts, this helical stent still has strong scaffolding properties. The greater diameter 120 and interior volume 115 present in the hollow interior of the helix configuration is illustrated in FIG. 8. As a result of this design, this device works well as a stent.

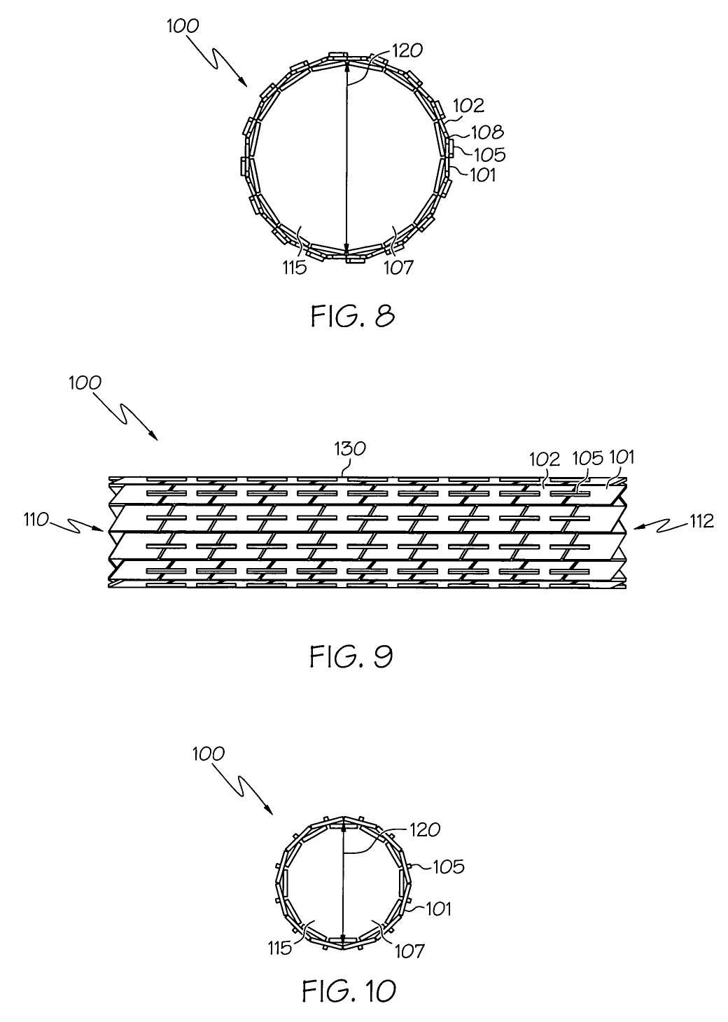

The geometrically shaped elements present in the stent can be any number or combination of shapes, making available a host of possible device configurations. These possible resulting configurations include spirals, helixes, cylinders, etc. and are well known to people of ordinary skill in the art. Given the elastic nature of the connectors, one possible embodiment of the invention is directed to a stent which changes from a cylindrical configuration as shown in FIGS. 5 and 9 to a helical configuration as shown in FIGS. 7 and 11 respectively. A number of possible embodiments of this invention are directed to a stent which has one geometric configuration in the unexpanded state and a different geometric configuration in the expanded state.

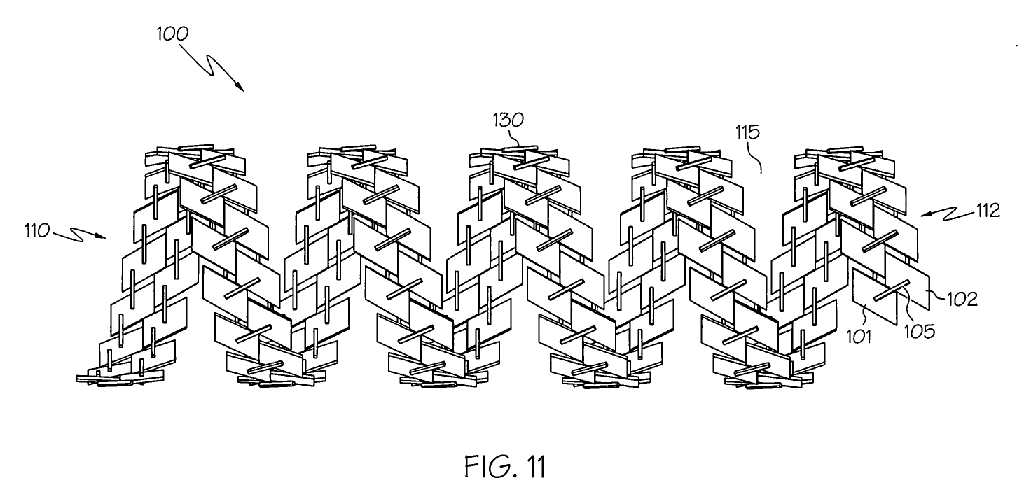

In at least one embodiment, an example of which is shown in FIG. 9, a stent 100 is provided with a cylinder configuration, and is constructed out of quadrilateral shaped elements 101 and 102. FIG. 10 illustrates this same configuration seen from the proximal side. This quadrilateral based configuration will also react to an energy input or strain (such as the expansion of a balloon) by changing the relative position of the quadrilateral elements which will change the device's overall configuration. As indicated above the shape and relative position of the elements 101 and 102 may be varied to provide the stent 100 with any of a variety of configurations and/or characteristics.

One possible configuration is illustrated in FIG. 11 where a stent 100 is made out of quadrilateral shaped elements 101 and 102 which combine to form a helical configuration. The helix configuration in FIG. 11 also has a hollow interior 115 with a greater diameter 120 and a greater volume than that found in the cylinder of FIG. 9.

The stent may be of uniform diameter in the unexpanded and expanded states or may be tapered in one or more states. Thus for example, the stent may be constructed and arranged to have a taper in one or more locations in an expanded configuration. The taper may be achieved in stent in which all of the geometric shaped elements are of the same shape by reorienting some of the geometric shaped elements by a first amount and others of the geometric shaped elements to a greater extent. The taper may also be achieved by using first geometric shaped elements of a first number of sides in one portion of the stent and second geometric shaped elements of a second number of sides in another portion of the stent. The stent may taper substantially continuously along the length of the expanded state or only section(s) of the stent may taper. The stent may be provided in a dumbbell shaped embodiment. More generally, one or more portions of the stent may assume a larger diameter than one or more other sections of the stent. The taper or different diameters may also be provided in an unexpanded embodiment.

The geometric shaped elements may be made of metal and/or polymer. Suitable metals include, but are not limited to, stainless steel, titanium and tantalum. Shape memory metals such as Nitinol may also be used. Other shape memory alloys may also be used. Other suitable metals include Elgiloy, and NP35N. Typically, the metals will be bio-compatible. It is also within the scope of the invention for the geometric shapes to be made of metal which is coated with one or more polymeric coatings or layers.

Suitable polymers include polyurethane, polystyrene, peek—polyaryletherketones, polyisobutylene copolymers, and styrene-isobutylene-styrene block copolymers such as styrene-isobutylene-styrene, tert-block copolymers (SIBS), polyvinylpyrrolidone including cross-linked polyvinylpyrrolidone, polyvinyl alcohols, copolymers of vinyl monomers such as EVA, polyvinyl ethers, polyvinyl aromatics, polyethylene oxides, polyesters including polyethylene terephthalate, polyamides, polyacrylamides, polyethers including polyether sulfone, polyalkylenes including polypropylene, polyethylene and high molecular weight polyethylene, polyurethanes, polycarbonates, silicones, siloxane polymers, cellulosic polymers such as cellulose acetate, polymer dispersions such as polyurethane dispersions, squalene emulsions, and mixtures and copolymers of any of the foregoing.

As can be seen in FIGS. 6, 7, 8, 9, 10 and 11, the stent 100 not only creates a hollow interior cavity 115 but the geometrically shaped elements provide the stent's shaft significant surface area. This surface area can be coated with one or more therapeutic agents or medications and will act as a large reaction surface which will facilitate chemical or drug kinetics. Different agents, chemicals or drugs can be applied to the outer and inner surfaces to facilitate different therapies or reactions.

Some suitable therapeutic agents may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

As can be seen from the illustrations, by constructing a stent out of multiple geometrically shaped elements and by using connectors that are elastic enough to alter the relative positions of the geometrically shaped elements but strong enough to hold them together, a stent or catheter with high flexibility, strong scaffolding strength, and useful reaction properties can be constructed. Because of the light mass and thickness of the device, it can maintain a low profile and is highly versatile. As a result, this design addresses some of the shortcomings present in unibody stent designs.

Although illustrated and described herein with reference to certain specific embodiments, the present invention is nevertheless not intended to be limited to the details shown. Rather, various modifications may be made in the details within the scope and range of equivalents of the claims and without departing from the spirit of the invention. For example, a stent such as stent 100 shown and described herein may be configured to have any of a variety of shapes and configurations, which may vary between non-deployed and deployed states in addition the helical and/or tubular configurations shown and described.

The invention has been discussed above with regard to stents. More generally, the invention discussed above and below is directed to stents, grafts, stent-grafts, vena cava filters, expandable frameworks and similar implantable medical devices. The invention is also directed to catheter shafts and guidewires for use in medical devices. The medical device will typically be tubular.

In some embodiments, the medical device comprises a plurality of plates. Each plate has an inner surface and an outer surface and a plurality of sides disposed about the periphery of the plate extending between the inner and outer surfaces. At least two plates which are adjacent one another are connected flexibly one to the other to allow for reorientation of the plates relative to one another. Adjacent plates have a first orientation in which one of the sides of one of the plates abuts one of the sides of the plate adjacent thereto and a second orientation in which another of the sides of one of plates abuts the same or different side of the plate adjacent thereto.

In at least one embodiment, each of the plates is adjacent another plate and is flexibly interconnected to at least one plate adjacent thereto so as to allow for reorientation of the plates relative to one another. Each two adjacent plates have a first orientation in which one of the sides of one of the plates abuts one of the sides of the plate adjacent thereto and a second orientation in which another of the sides of one of plates abuts the same or different side of the plate adjacent thereto.

In some embodiments, the invention is directed to the medical device in the fully expanded state, the unexpanded state and in any states therebetewen. Typically, the medical device will be characterized as having a plurality of discrete expanded states with the stent having a different diameter in each of the discrete expanded states.

The plates may be provided a wide variety of shapes including polygonal and desirably, regular polygonal.

Each plate may be flexibly connected to a plate adjacent thereto via a flexible connector made of metal or polymer. The use of elastic connectors has been disclosed above. More generally, flexible connector connectors may be used. Any of the material disclosed herein for the geometric shaped elements may also be used for the flexible connectors. The flexible connectors may be elastic. It is also within the scope of the invention for the flexible connectors to be made of a material which can plastically deform.

The flexible materials may be made of biodegradable materials, for example biodegradable polymers as are know in the art. Examples of suitable biodegradable materials include fibrin, collagen, polymers, polyurethane, sugars, polyanhydrides, polyethyloxides, polycarboxylic acid, polyanhydrides including maleic anhydride polymers, polyorthoesters, poly-amino acids, polyethylene oxide, polyphosphazenes, polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide), polydioxanone, polypropylene fumarate, polydepsipeptides, polycaprolactone, and co-polymers and mixtures thereof such as poly(D,L-lactide-co-caprolactone) and polycaprolactone co-butylacrylate, polyhydroxybutyrate valerate and blends, polycarbonates such as tyrosine-derived polycarbonates and arylates, polyiminocarbonates, and polydimethyltrimethylcarbonates, cyanoacrylate, calcium phosphates, polyglycosaminoglycans, macromolecules such as polysaccharides (including hyaluronic acid, cellulose, and hydroxypropylmethyl cellulose, gelatin, starches, dextrans, alginates and derivatives thereof), proteins and polypeptides, and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc calcium phosphate.

Biodegradable materials may be mixed with therapeutic substances, if desired, for release into the body lumen upon biodegradation of the material

The plates may be arranged substantially helically about a longitudinal axis or in closed cylinders about a longitudinal axis.

An example of a suitable tubular medical device formed in accordance with the invention is shown at 100 in FIG. 5. The plates are shown in the form of octagonal elements at 101 and 102.

In at least one embodiment, the invention is also directed to a stent comprising a plurality of stent segments, at least two adjacent segments having a plurality of meshing teeth to allow one of the segments to be repositioned relative to the other of the two segments. Desirably, each segment is engaged to at least one adjacent segment via meshing teeth to allow the segment to be repositioned relative to the segment to which it is engaged.

The stent will desirably be expandable between at least two sizes. More desirably, the stent will be discontinuously expandable between more that two different, discrete sizes.

The segments may be arranged helically about a longitudinal axis or may be arranged in serpentine bands which form closed cylinders.

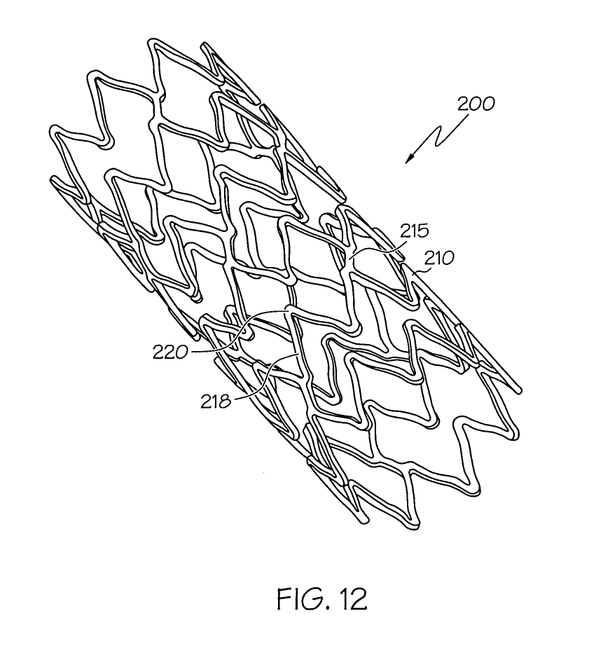

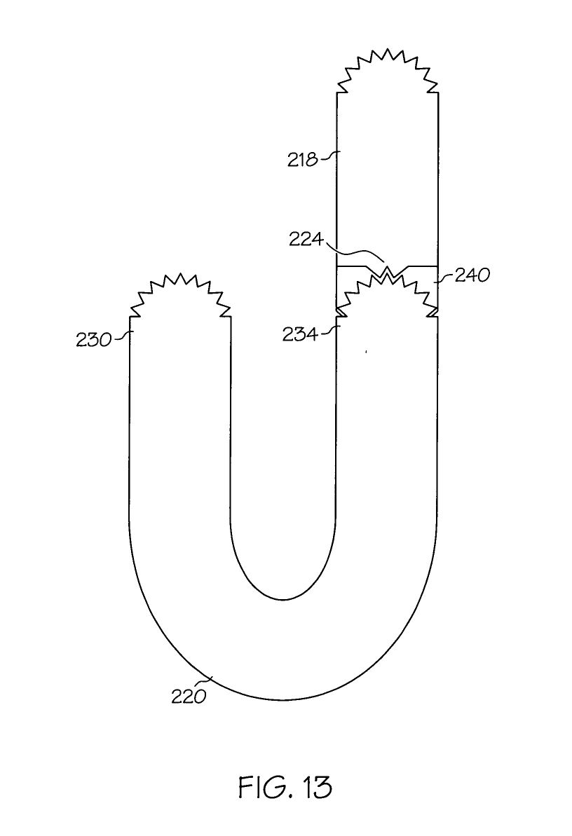

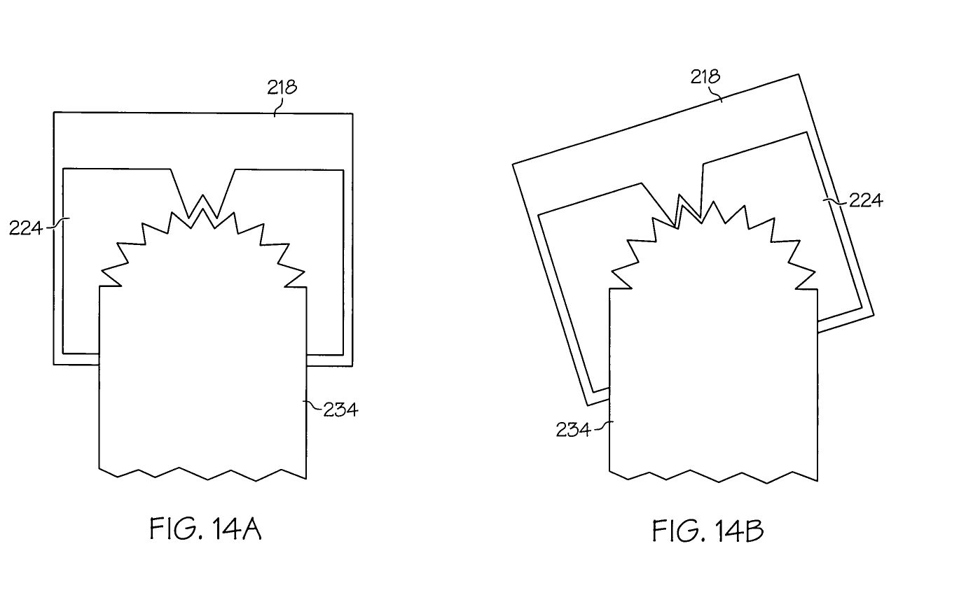

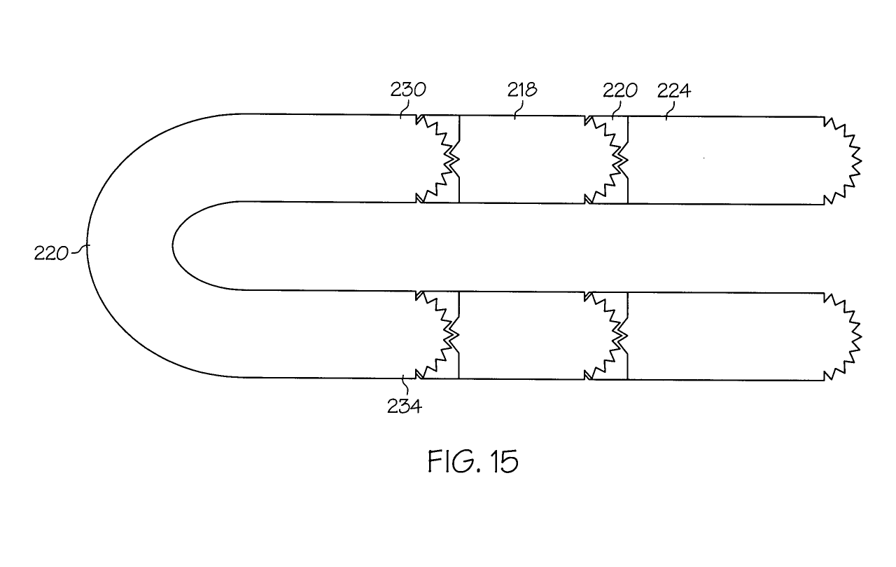

An example of such a stent is shown schematically at 200 in FIG. 12 and in more detail in FIGS. 13, 14a and 14b. FIG. 12 shows a stent comprising a plurality of serpentine bands, 210 which are connected via connectors 215. Serpentine bands 210 comprise a plurality of strut 218 connected by turns 220. FIG. 13 illustrates a turn 220 and a strut 218 extending therefrom. Turn 220 includes a portion with a plurality of teeth 224 extending therefrom from a first end 230 and a second end 234. Teeth 224 mate with complementary teeth 240 on strut 218. As shown in FIG. 13, strut 218 includes teeth at both ends. FIGS. 14a and 14b show enlargements of FIG. 13. FIG. 15 shows a schematic of a turn 220 with a plurality of strut segments extending therefrom. Adjacent segments are interconnected by meshed teeth so that the strut segments and turns may be reoriented relative to one another. Optionally, the connectors between serpentine bands may also have teeth and may be constructed to matingly engage with a portion of the serpentine band which has teeth. Although the connectors 215 are shown as being straight with ends which are circumferentially and longitudinally offset from one another, the connectors may be curved, having or more curved portions or straight and the ends may be circumferentially offset and/or longitudinally offset or aligned. Individual sections of the connector may be provided with meshing teeth so that the connector can reorient, as necessary. Such a stent, or more generally, medical device, may be made in the form of cylindrical bands as shown in FIG. 12 or may be in the form of a helical stent. The invention may also be applied to stents having overlapping circumferential bands which lack connectors. FIGS. 14a and 14b show one configuration of meshing teeth. Other configurations are also within the scope of the invention.

Any of the stents disclosed herein may be provide with a cover or liner, typically of a polymeric material, over portions or the entirety of the device to form a grafts or stent-grafts. The plates or geometric shaped elements or members with meshing teeth may also be configured to form vena cava filters, expandable frameworks and similar implantable medical devices.

The designs disclosed herein may also be applied to the construction of tubes for catheter. Thus, for example, the plates or segments with teeth may be assembled into tubes having diameters which render the tubes suitable for use as catheter tubes. The catheter tube may be provided with a variable diameter. Moreover, the stiffness of the catheter may be varied by transitioning the catheter tube between a wider diameter (less stiff) and a narrower diameter (stiffer) configuration.

Other suitable medical devices which fall within the scope of the invention include guidewires constructed using the techniques disclosed herein for making tubes.

The invention is also directed to the inventive medical devices disclosed herein in combination with a delivery catheter and, where appropriate, disposed on, in or about a delivery catheter. Thus, for example, the invention is directed to stents, grafts, stent-grafts, vena cava filters, expandable frameworks and similar implantable medical devices disposed on, about or in a delivery catheter.

At least one embodiment of the invention is further directed to a method of treating a bodily location using any of the medical devices disclosed herein. Thus, by way of example, the invention is directed to a method of treating a bodily vessel comprising the steps of disposing an inventive medical device on, in or about a delivery device such as a catheter. The medical device may be a stent, stent-graft, graft, vena cava filter, expandable framework or any other implantable medical device disclosed herein. The medical device is delivered to a desired bodily location and the medical device deployed, typically by expansion, whether self-expansion or balloon initiated expansion. The delivery device may then be withdrawn from the body.

One such method of treating a blood vessel or other bodily vessel or channel comprises the steps of: providing a stent having a generally tubular body, the tubular body defining an interior. The stent has an expanded and unexpanded configuration and comprises at least two geometric shaped elements, desirably, polygonal shaped. Each shaped element has a plurality of sides, an outer surface, an inner surface, and at least one locking point and at least one connector, desirably elastic, connecting the geometric shaped elements. In the unexpanded configuration, at least two of the geometric shaped elements are positioned adjacent to each other in a first relative position and in the expanded configuration, the stent assumes a configuration in which the two geometric shaped elements assume a second relative position in which at least two of the locking points fit into each other and lock the two geometric shaped elements together. The tension induced by the connector pulls the geometric shaped elements together to keep them in the expanded configuration in an unexpanded configuration. The unexpanded stent is disposed on, in or about a catheter and inserted into a blood vessel. The stent is then deployed and the catheter withdrawn.

Grafts, stent-grafts, vena-cava filters and any other tubular, implantable medical device may be similarly delivered and placed in a vessel.

Some embodiments of the invention are also directed to methods of delivering a catheter to a desired bodily location by using an inventive catheter tube disclosed herein, inserting at least a portion of the inventive catheter tube into a bodily vessel and changing the diameter and/or stiffness of the catheter tube by reorienting the plates or polygonal geometric shaped elements relative to one another so that the catheter tube achieves a larger diameter or smaller diameter cross-section.

The invention is also directed to method of manufacturing any of the inventive medical devices disclosed herein by providing a plurality of plates or polygonal, geometric shaped elements and flexibly interconnected the elements to form a tube.

Although the inventive devices disclosed above have been described as medical devices, one of ordinary skill in the art will recognize that the tubes disclosed herein may also be used for non-medical applications in which tubes or pipes are required, although they may, in certain applications, larger diameters may be required. To that end, the invention is further directed to any of the inventive tubes disclosed herein sized for use in other industries, for example, in the plumbing, electrical and oil field industries.

This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A stent having a generally tubular body, the tubular body defining an interior, the stent, comprising at least two polygonal geometrically shaped elements, each element having a plurality of sides, an outer surface, and inner surface, and at least one elastic connector connecting two of the polygonal geometrically shaped elements such that the polygonal geometric shaped elements can assume a plurality of stable orientations relative to one another

2. The stent of claim 1 wherein the sides of at least one polygonal geometrically shaped elements define an octagonal shape.

3. The stent of claim 1 wherein the sides of at least one polygonal geometrically shaped elements define a rectangular shape.

4. The stent of claim 1 wherein the polygonal geometrically shaped elements combine to form a cylindrical configuration.

5. The stent of claim 1 wherein the polygonal geometrically shaped elements combine to form a helical configuration.

6. The stent of claim 1 wherein at least one of the connectors is made out of metal.

7. The stent of claim 1 wherein at least one of the connectors is made out of polymer.

8. The stent of claim 1 wherein at least one of the geometrically shaped elements is made out of metal.

9. The stent of claim 1 wherein at least one of the polygonal geometrically shaped elements is made out of a polymer selected from the group consisting of polystyrene, styrene-isobutylene-styrene block copolymers, polyvinylpyrrolidone, copolymers of vinyl monomers polyethers, and polymer dispersions.

10. The stent of claim 1 wherein at least one of the polygonal geometrically shaped elements has medication applied to it.

11. The stent of claim 1 having an unexpanded configuration and at least one expanded configuration.

12. The stent of claim 12 wherein in the unexpanded state, at least two polygonal geometrically shaped elements overlap each other.

13. The stent of claim 12 wherein in the unexpanded state, at least two polygonal geometrically shaped elements are adjacent to each other.

14. The stent of claim 12 wherein in the unexpanded state, at least two polygonal geometrically shaped elements are in a locked position.

15. The stent of claim 12 wherein in the unexpanded state, at least two polygonal geometrically shaped elements are in an unlocked position.

16. The stent of claim 12 wherein when the stent changes from an unexpanded state to an expanded state, the stent configuration changes from a cylindrical configuration to a helical configuration.

17. The stent of claim 12 wherein in the expanded state the stent has the same general shape as in the unexpanded state.

18. A stent having a generally tubular body, the tubular body defining an interior,
the stent having an expanded and unexpanded configuration and comprising at least two octagonal shaped elements,
each shaped element having a plurality of sides, an outer surface, an inner surface, and at least one locking point and at least one elastic connector connecting the octagonal shaped elements wherein:
in the unexpanded configuration at least two of the octagonal shaped elements are positioned adjacent to each other in a first relative position forming stent in a cylindrical configuration and
in the expanded configuration, stent assumes a cylindrical configuration in which the two octagonal shaped elements assume a second relative position in which at least two of the locking points fit into each other and lock the two octagonal shaped elements together, and the tension induced by the elastic connector pulls the octagonal shaped elements together to keep them in the expanded configuration.

19. A method of treating a blood vessel comprising the steps of:
providing a stent having a generally tubular body, the tubular body defining an interior, the stent having an expanded and unexpanded configuration and comprising at least two geometric shaped elements, each shaped element having a plurality of sides, an outer surface, an inner surface, and at least one locking point and at least one elastic connector connecting the geometric shaped elements wherein: in the unexpanded configuration at least two of the geometric shaped elements are positioned adjacent to each other in a first relative position and in the expanded configuration, stent assumes a configuration in which the two geometric shaped elements assume a second relative position in which at least two of the locking points fit into each other and lock the two geometric shaped elements together, and the tension induced by the elastic connector pulls the geometric shaped elements together to keep them in the expanded configuration in an unexpanded configuration;
emplacing the unexpanded stent on a catheter;
inserting the stent and catheter into a blood vessel; and
deploying the stent.

20. A stent comprising a plurality of plates, each plate having an inner surface and an outer surface and a plurality of sides disposed about the periphery of the plate extending between the inner and outer surfaces, at least two plates which are adjacent one another connected flexibly one to the other to allow for reorientation of the plates relative to one another, adjacent plates having a first orientation in which one of the sides of one of the plates abuts one of the sides of the plate adjacent thereto and a second orientation in which another of the sides of one of plates abuts the same or different side of the plate adjacent thereto.

21. The stent of claim 20 wherein each of the plates is adjacent another plate, each plate flexibly interconnected to at least one plate adjacent thereto so as to allow for reorientation of the plates relative to one another, each two adjacent plates having a first orientation in which one of the sides of one of the plates abuts one of the sides of the plate adjacent thereto and a second orientation in which another of the sides of one of plates abuts the same or different side of the plate adjacent thereto.

22. The stent of claim 21 wherein the stent can expand from an unexpanded state to an expanded state.

23. The stent of claim 21 characterized as having a plurality of discrete expanded states, the stent having a different diameter in each of the discrete expanded states.

24. The stent of claim 21 wherein the plates are each polygonal in shape.

25. The stent of claim 24 wherein the plates are each in the form of a regular polygon.

26. The stent of claim 24 wherein the plates are octagonal in shape.

27. The stent of claim 20 wherein each plate is flexibly connected to a plate adjacent thereto via a flexible connector made of metal.

28. The stent of claim 20 wherein each plate is flexibly connected to a plate adjacent thereto via a flexible connector made of polymer.

29. The stent of claim 20 wherein the plates substantially helically about a longitudinal axis.

30. A stent comprising a plurality of stent segments, at least two adjacent segments having a plurality of meshing teeth to allow one of the segments to be repositioned relative to the other of the two segments.

31. The stent of claim 30 wherein each segment is engaged to at least one adjacent segment via meshing teeth to allow the segment to be repositioned relative to the segment to which it is engaged.

32. The stent of claim 31 wherein the stent is discontinuously expandable to a plurality of different, discrete sizes.

33. The stent of claim 31 wherein the segments are arranged helically about a longitudinal axis.

34. The stent of claim 31 wherein the segments are arranged in serpentine bands which form closed cylinders
